(19) United States
(12) Patent Application Publication
(10) Pub. No.: US 2024/0416091 A1
(43) Pub. Date: Dec. 19, 2024

(54) DUAL FRAME SHUNT

(71) Applicant: EDWARDS LIFESCIENCES CORPORATION, Irvine, CA (US)

(72) Inventor: Rani Abdullah Mahmoudi, Huntington Beach, CA (US)

(21) Appl. No.: 18/815,297

(22) Filed: Aug. 26, 2024

Related U.S. Application Data
(63) Continuation of application No. PCT/US2023/014392, filed on Mar. 2, 2023.
(60) Provisional application No. 63/318,545, filed on Mar. 10, 2022.

(51) Int. Cl. A61M 27/00

(52) U.S. Cl. CPC A61M 27/002; A61M 2210/125

(57) ABSTRACT

Systems, devices, and methods for shunting blood from one anatomical chamber/vessel to another anatomical chamber/vessel are described. For example, a dual frame shunt can include an anchoring frame/structure that is configured to self-expand and to anchor the shunt within an opening between two anatomical chambers/features. The shunt can also include an adjustable frame/structure disposed outside or inside of at least a portion of the anchoring frame and configured to expand/adjust to various sizes.

### RELATED APPLICATION

This application is a continuation of International Application No. PCT/US2023/014392, filed Mar. 2, 2023, and entitled DUAL FRAME SHUNT, which claims priority to U.S. Provisional Patent Application No. 63/318,545, filed on Mar. 10, 2022, and entitled DUAL FRAME SHUNT, the complete disclosures of which are hereby incorporated by reference in their entirety.

### BACKGROUND

### Field

The present disclosure generally relates to the field of medical implant devices.

### Description of Related Art

Certain physiological parameters associated with chambers of the heart, such as fluid pressure, can have an impact on patient health prospects. In particular, high cardiac fluid pressure can lead to heart failure and/or other complications in some patients. Therefore, reduction of pressure in certain chambers of the heart through blood flow shunting can improve patient health in some cases.

### SUMMARY

Described herein are one or more methods and/or devices to facilitate the shunting of blood between chamber(s)/vessel(s) of the heart or other anatomy.

In examples, the present disclosure relates to a shunt system comprising an anchoring frame configured to be disposed within an opening between a first anatomical feature and a second anatomical feature and expand from a collapsed state to an expanded state. The anchoring frame includes a self-expanding form. The shunt system further comprises an adjustable frame disposed around at least a portion of the anchoring frame and configured to adjust in size to set an amount of fluid that flows between the first and second anatomical features.

In some instances, the anchoring frame includes one or more arms configured to, in the expanded state, extend from a central portion of the anchoring frame to anchor the shunt system to a tissue wall.

In some instances, the adjustable frame comprises a permanently deformable form.

In some instances, the anchoring frame includes a shape-memory material.

In some instances, the adjustable frame includes at least one of stainless steel, cobalt chromium, or aluminum.

In some instances, at least one of the anchoring frame or the adjustable frame includes a covering disposed between the anchoring frame and the adjustable frame.

In some instances, the anchoring frame and the adjustable frame are independent frame structures that are coupled together with one or more attachment features.

In some instances, the adjustable frame includes a wire frame having a substantially cylindrical form.

In some instances, the anchoring frame includes a first anchoring arm configured to contact a first tissue wall within the first anatomical feature and a second anchoring arm configured to contact a second tissue wall within the second anatomical feature. The first anchoring arm is angularly offset from the second anchoring arm with respect to a longitudinal axis of the adjustable frame.

In some instances, at least one of the first anchoring arm or the second anchoring arm includes a visual marker configured to provide imaging visualization.

In examples, the present disclosure relates to a method of providing blood flow between a first anatomical feature and a second anatomical feature. The method comprises advancing a delivery catheter into an opening in a tissue wall between the first anatomical feature and the second anatomical feature and deploying, using the delivery catheter, a shunt within the opening. The shunt includes an anchoring frame configured to anchor the shunt to the tissue wall and an adjustable frame disposed around at least a portion of the anchoring frame. The anchoring frame is configured with at least one of shape memory or a super elasticity characteristic. The method further comprises dilating the adjustable frame to configure an amount of the blood flow between the first anatomical feature and the second anatomical feature.

In some instances, the method further comprises determining at least one of the amount of blood flow between the first and second features or pressure in the first anatomical feature or the second anatomical feature, and adjusting the amount of blood flow by further dilating the adjustable frame.

In some instances, the deploying the shunt, the dilating the adjustable frame, the determining, and the adjusting the amount of blood flow occur during the same medical procedure.

In some instances, the first anatomical feature is a left atrium of a heart and the second anatomical feature is at least one of a coronary sinus or a right atrium.

In some instances, the adjustable frame comprises a permanently deformable form.

In some instances, the adjustable frame includes at least one of stainless steel, cobalt chromium, or aluminum.

In some instances, at least one of the anchoring frame or the adjustable frame includes a covering disposed between the anchoring frame and the adjustable frame.

In some instances, the anchoring frame and the adjustable frame are independent frame structures that are coupled together with one or more attachment features.

In some instances, the deploying the shunt includes releasing a first arm of the anchoring frame to contact a first tissue wall within the first anatomical feature and releasing a second arm of the anchoring frame to contact a second tissue wall within the second anatomical feature.

In examples, the present disclosure relates to a shunt system comprising a first frame structure configured to anchor the shunt system to an opening between a first anatomical feature and a second anatomical feature. The first frame structure is configured to implement a compressed form on a delivery system and to self-expand to an at least partially expanded form when released from the delivery system. The shunt system further comprises a second frame structure coupled to at least a portion of the first frame structure. The second frame structure being configured to radially expand, based at least in part on a radial force from a dilator, to adjust an amount of fluid flow between the first and second features.

In some instances, the first frame structure includes a first anchoring arm configured to contact a first tissue wall within the first anatomical feature and a second anchoring arm configured to contact a second tissue wall within the second anatomical feature. The first anchoring arm is angularly offset from the second anchoring arm with respect to a longitudinal axis of the second frame structure.

In some instances, the second frame structure comprises a permanently deformable form.

In some instances, the second frame structure includes at least one of stainless steel, cobalt chromium, or aluminum.

In some instances, at least one of the first frame structure or the second frame structure includes a covering disposed between the first frame structure and the second frame structure.

In some instances, the first frame structure and the second frame structure are independent frame structures that are coupled together with one or more attachment features.

In some instances, the shunt system is sterilized.

For purposes of summarizing the disclosure, certain aspects have been described. It is to be understood that not necessarily all such advantages may be achieved in accordance with any particular example. Thus, the disclosed examples can be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the subject matter. In addition, various features of different disclosed examples can be combined to form additional examples, which are part of this disclosure. Throughout the drawings, reference numbers may be reused to indicate correspondence between reference elements.

FIG. 1 illustrates human cardiac anatomy in accordance with one or more examples.

FIG. 2 illustrates a section of the heart from a top-down, superior perspective with the posterior aspect oriented at the top of the page in accordance with one or more examples.

FIG. 3A illustrates a perspective view of a shunt in a configuration with an adjustable frame positioned around a portion of an anchoring frame according to one or more examples.

FIG. 313 illustrates a side view of the shunt of FIG. 3A according to one or more examples.

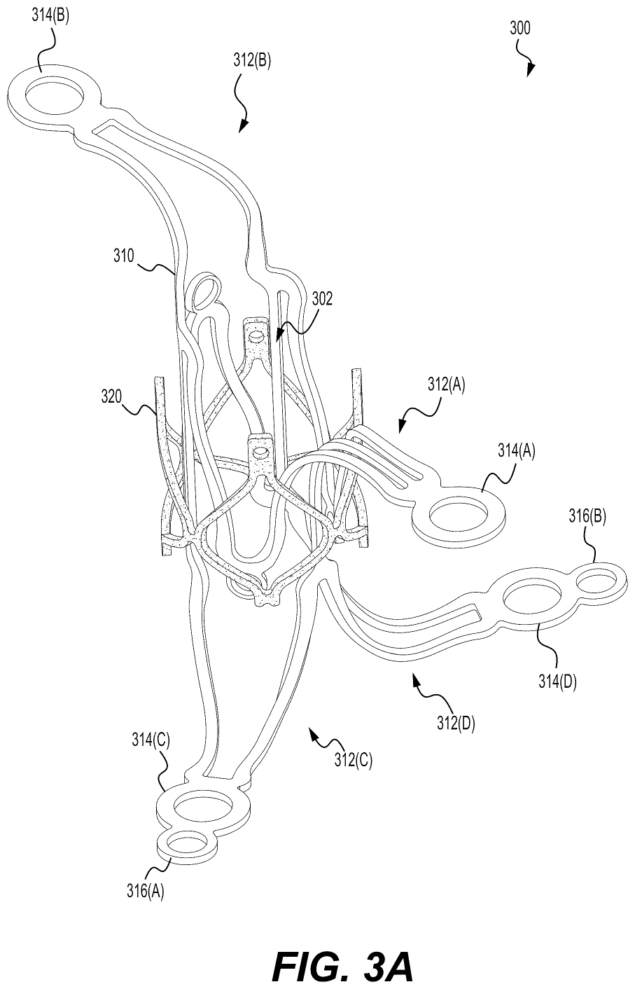

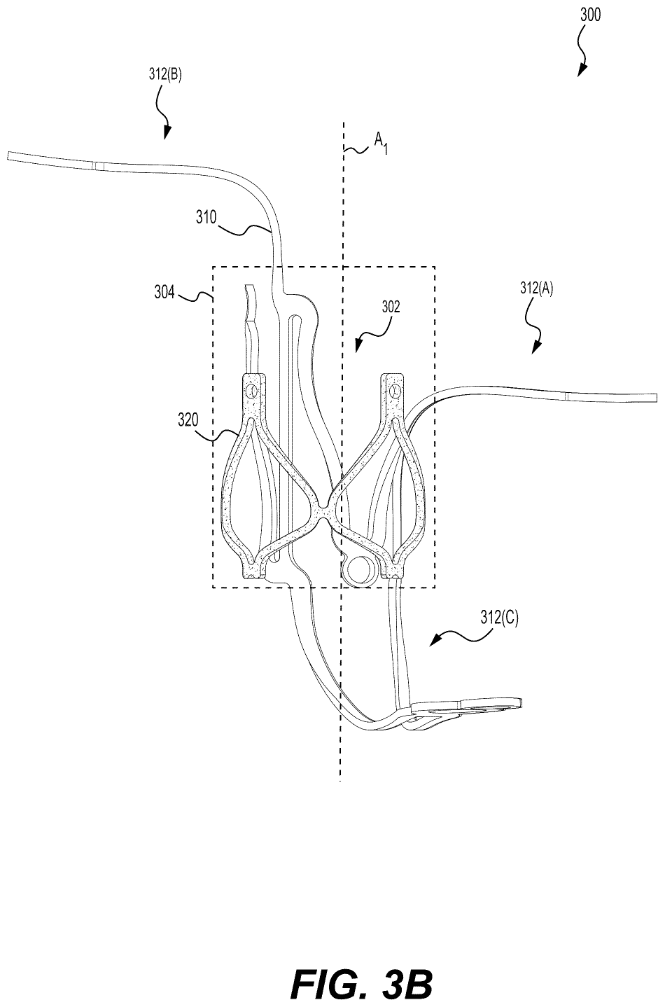

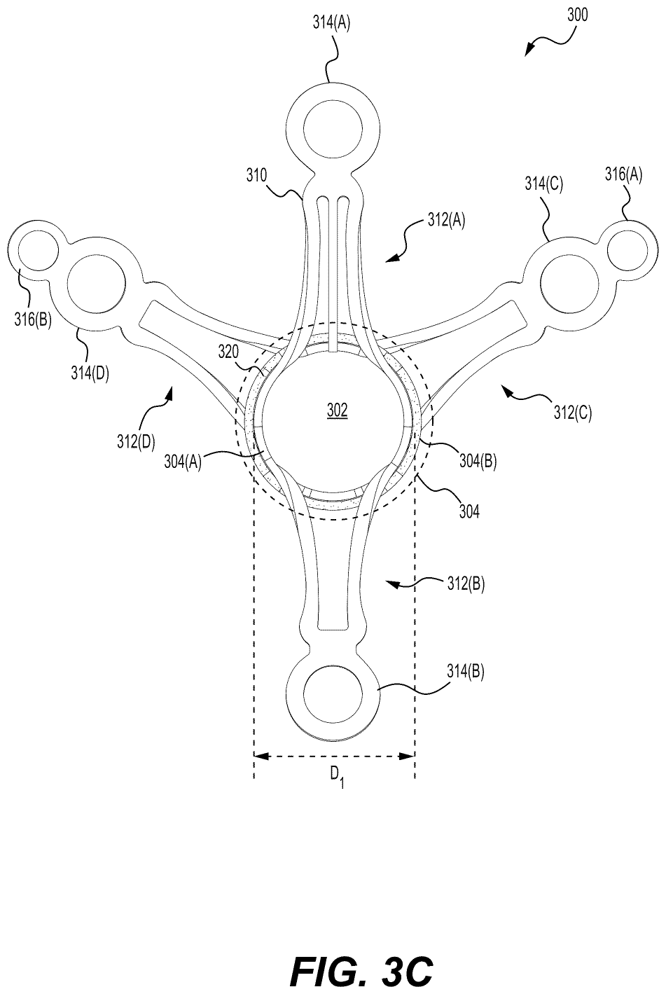

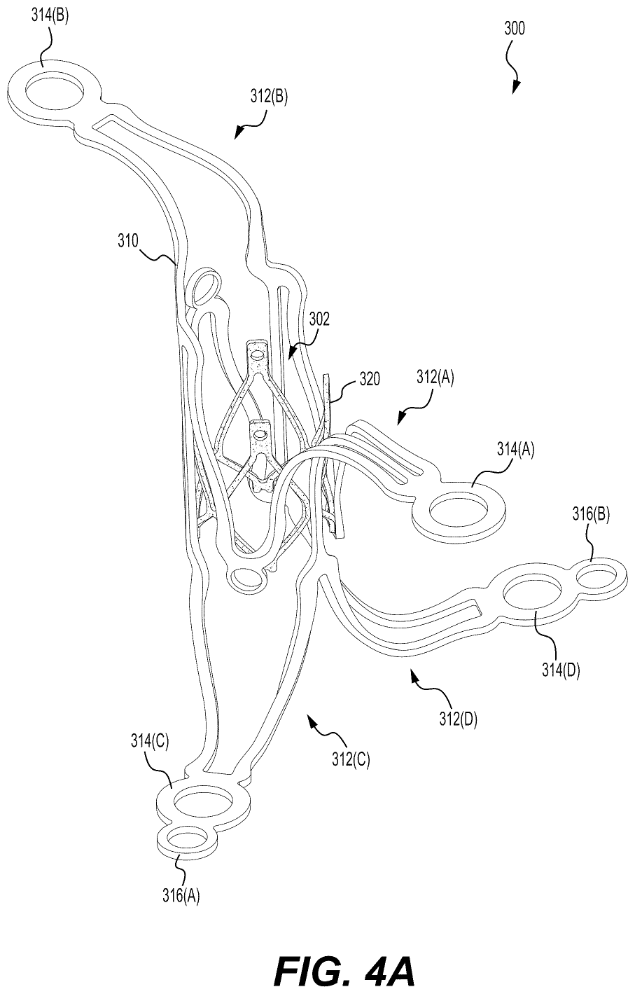

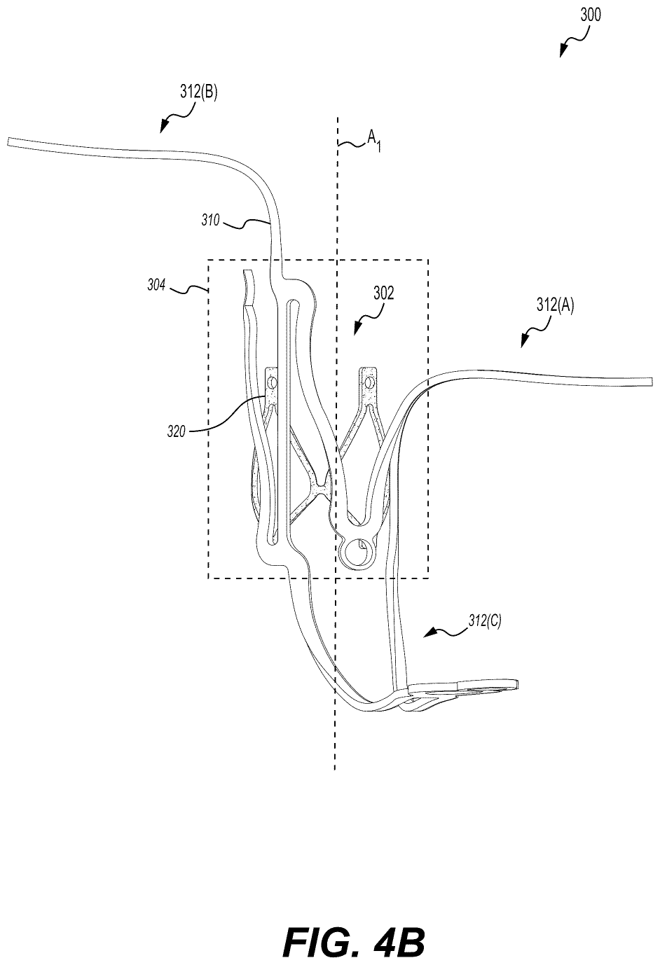

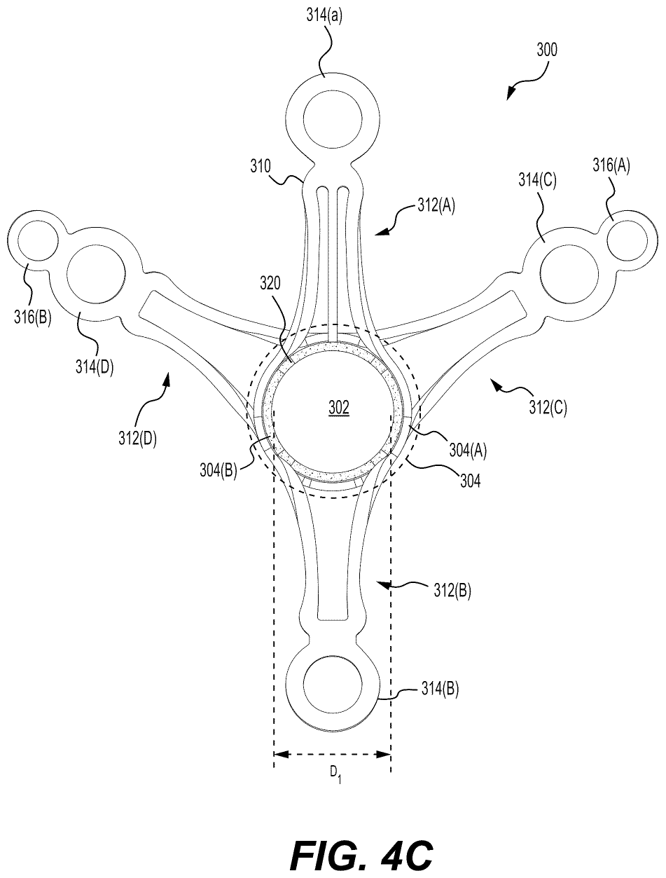

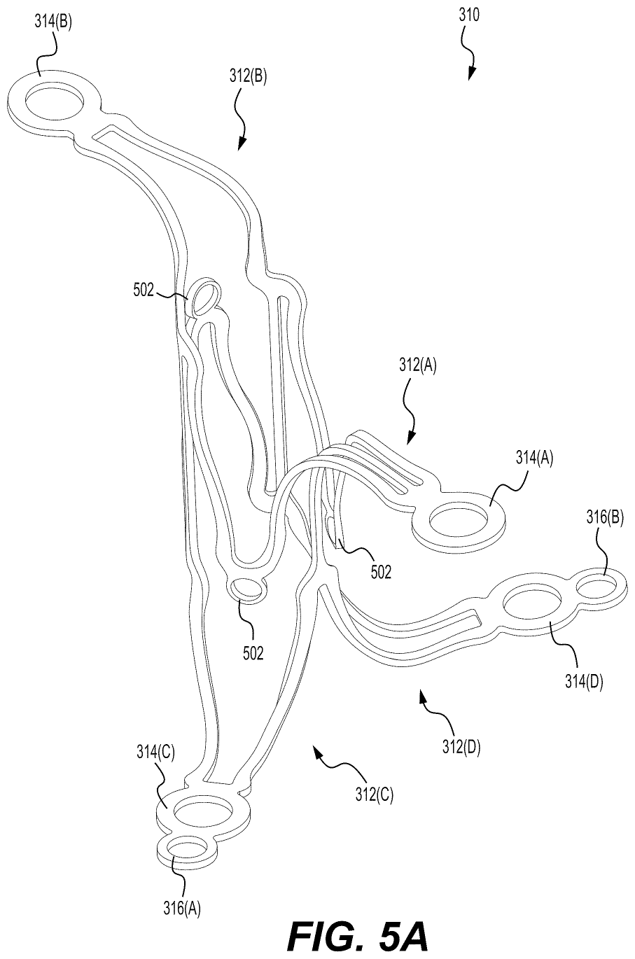

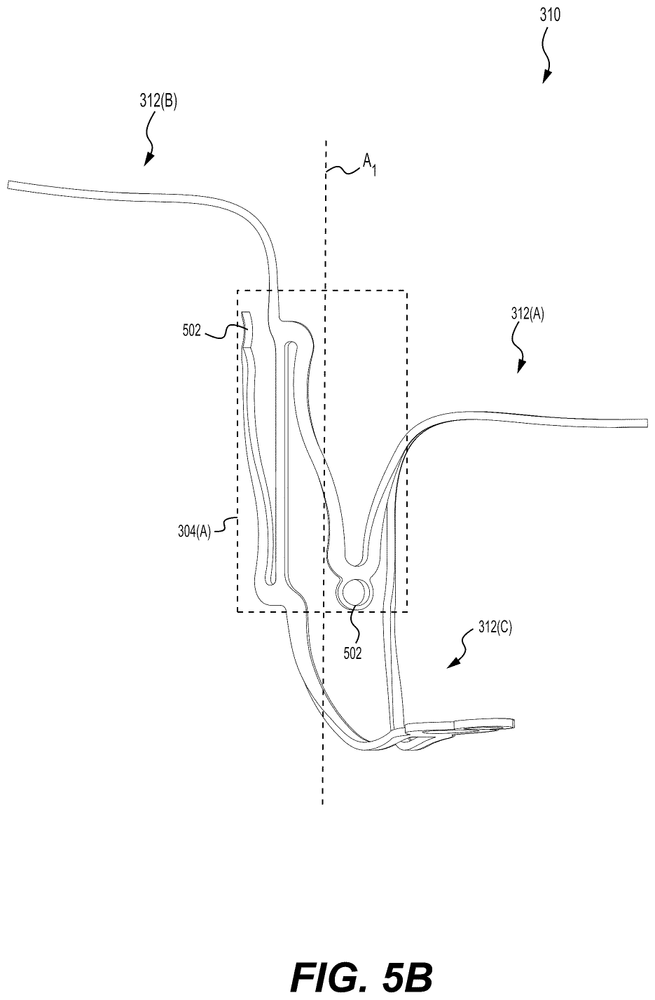

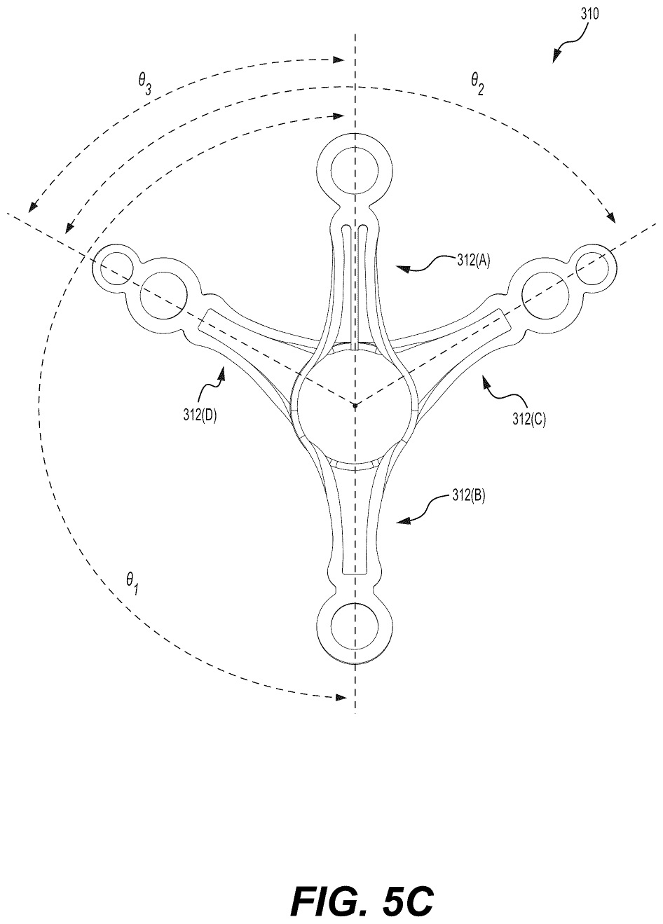

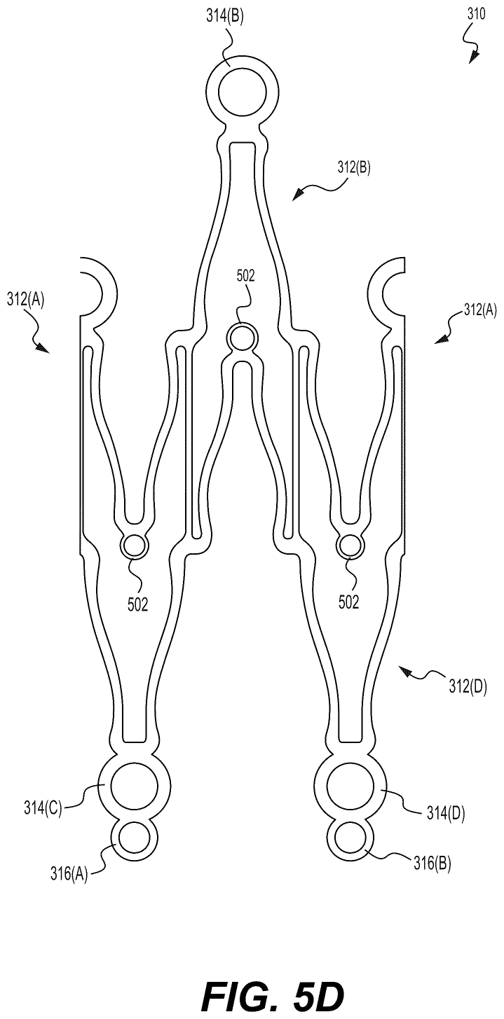

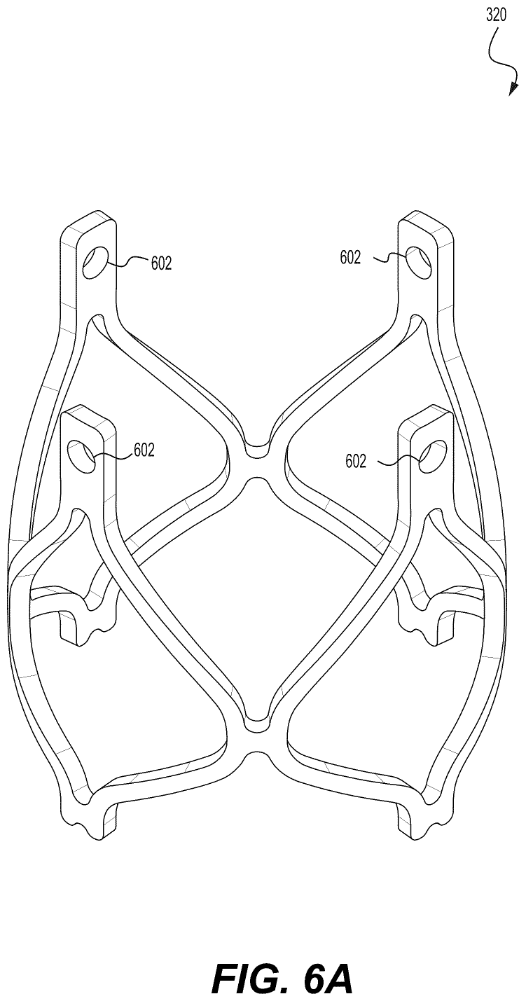

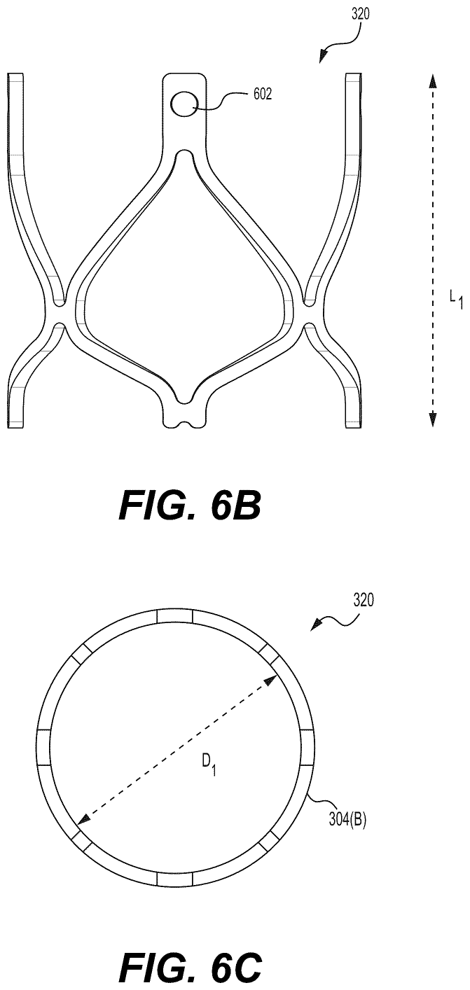

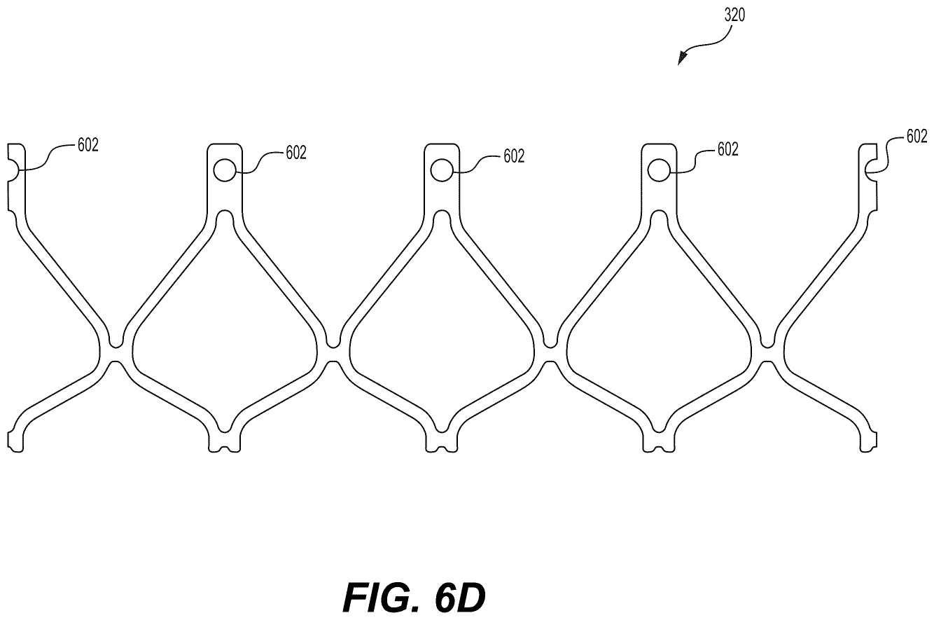

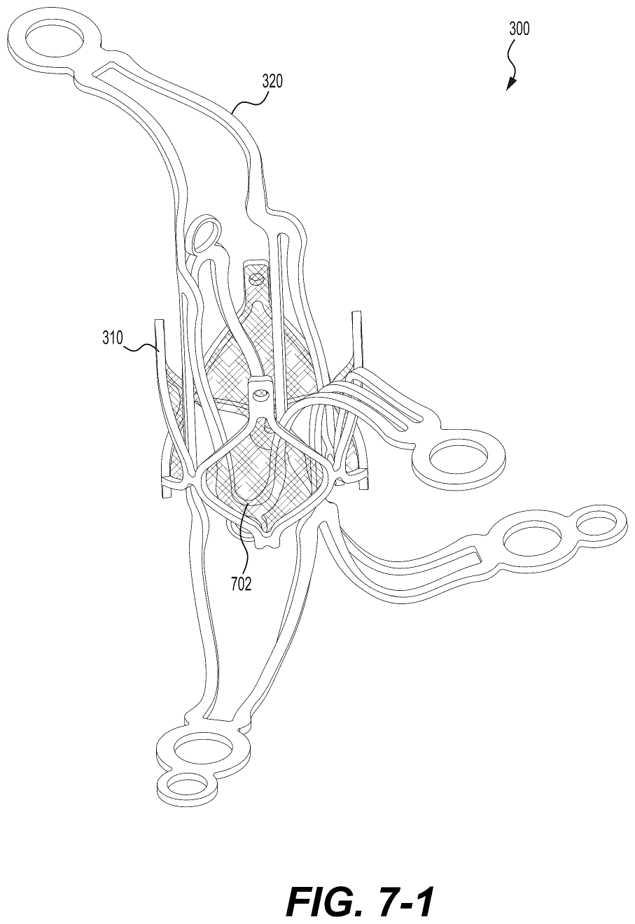

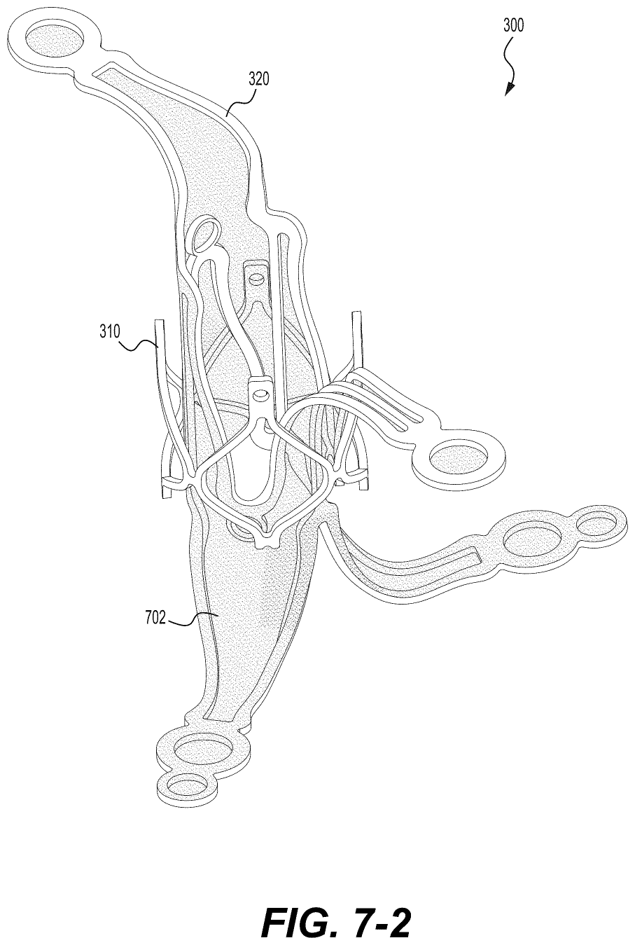

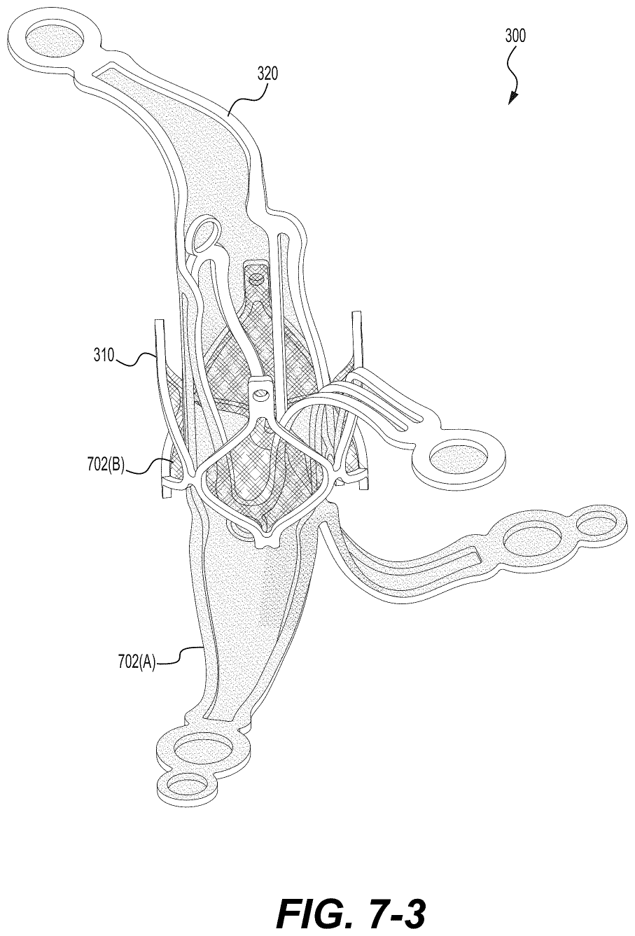

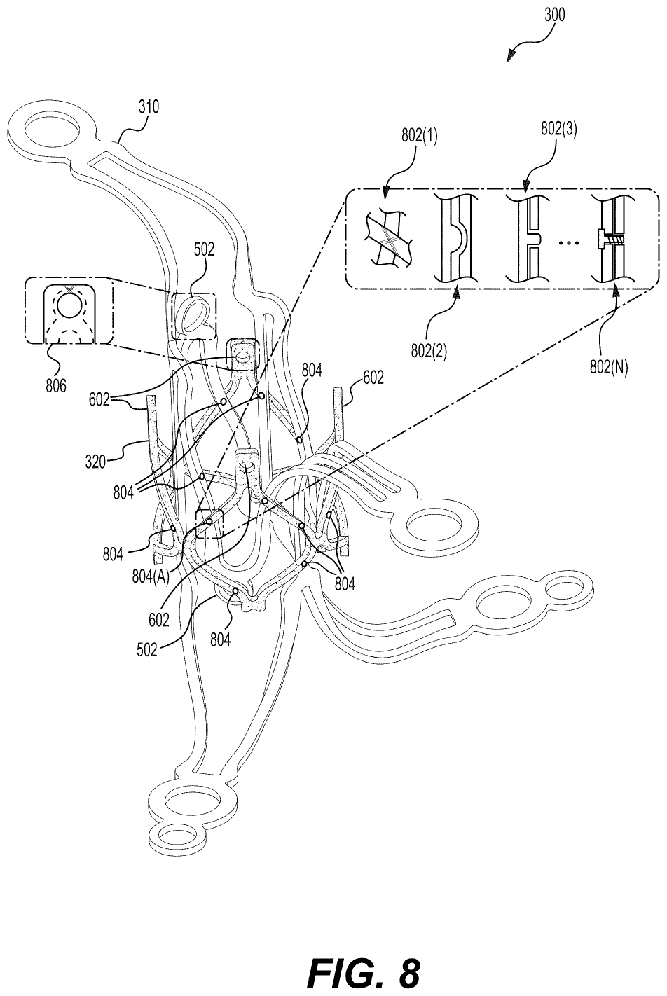

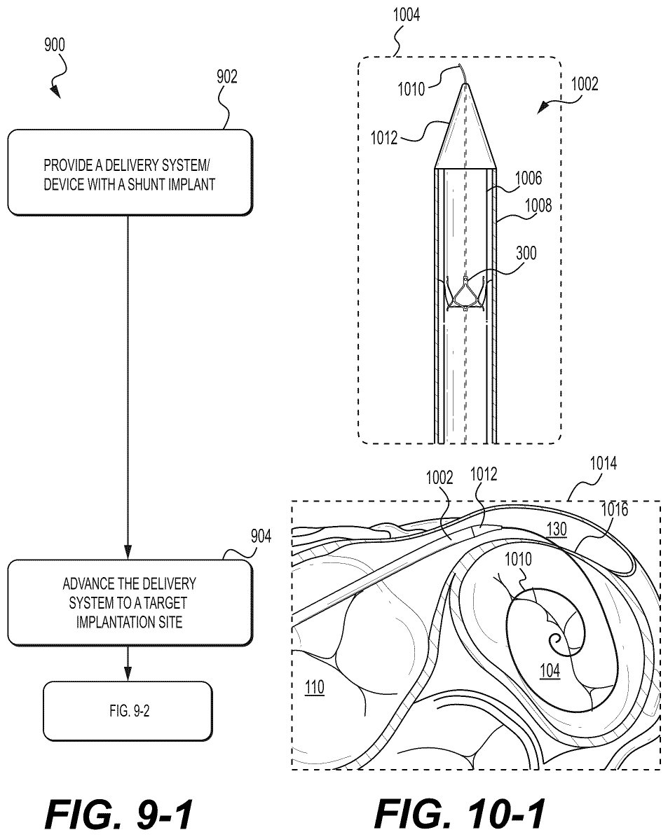

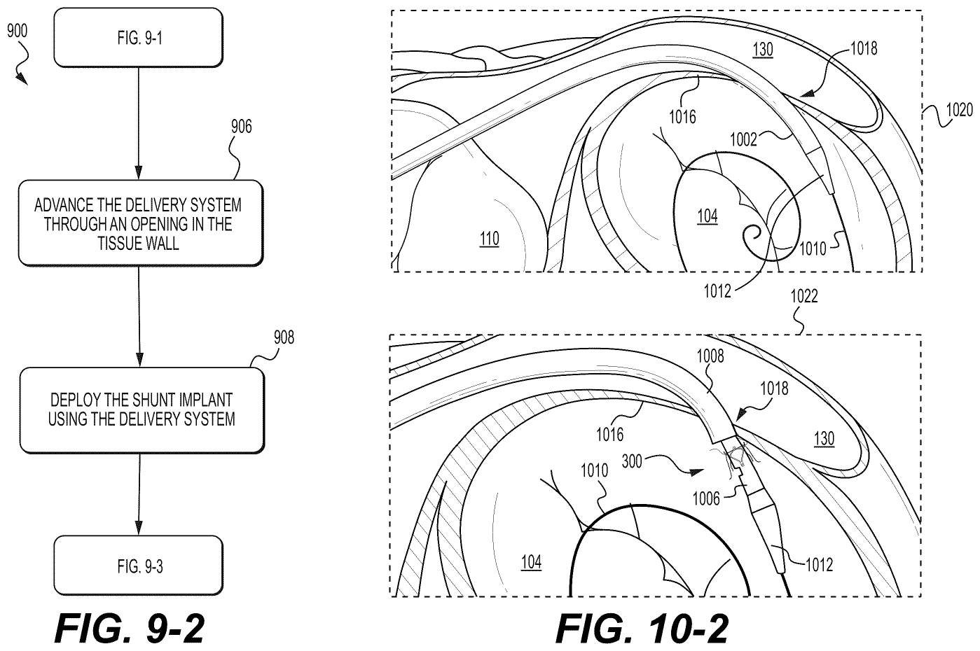

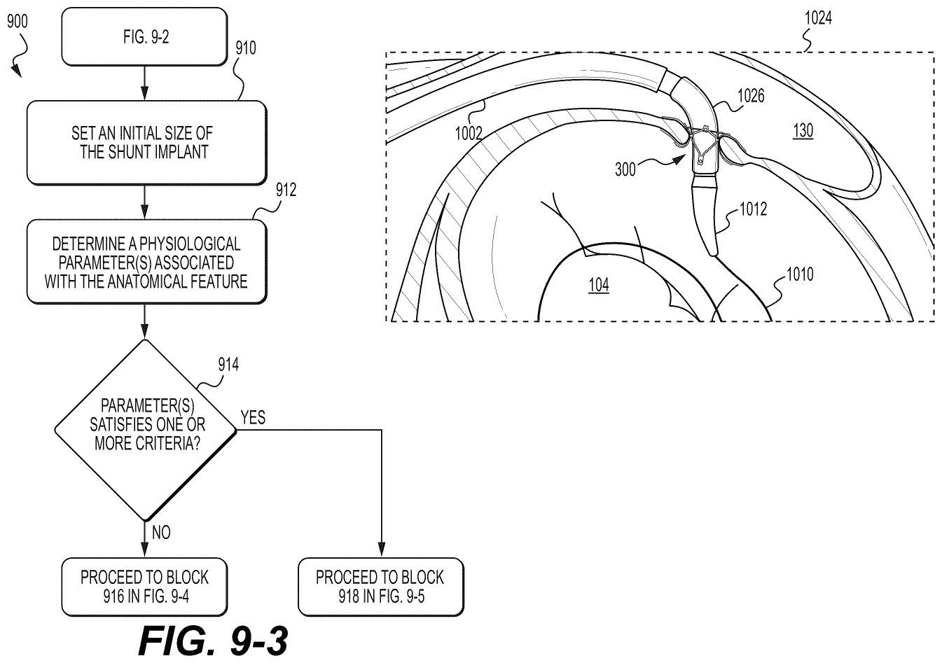

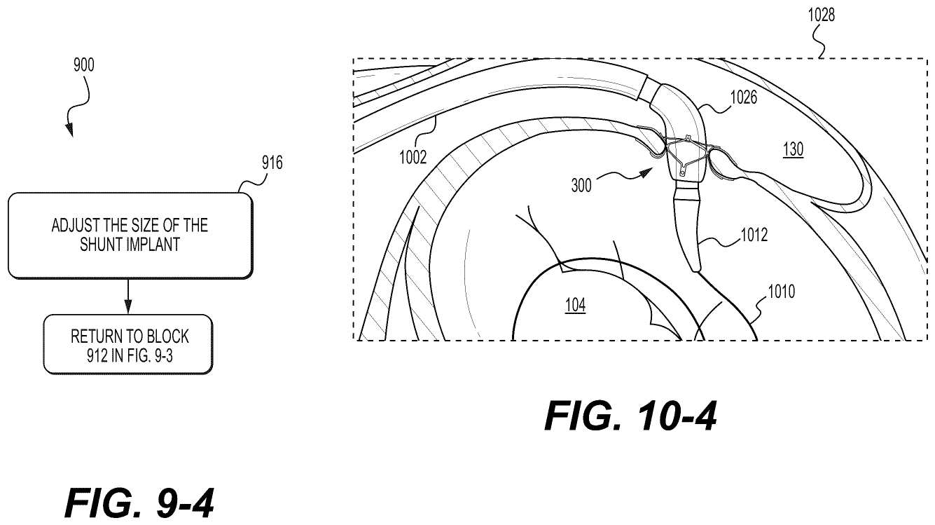

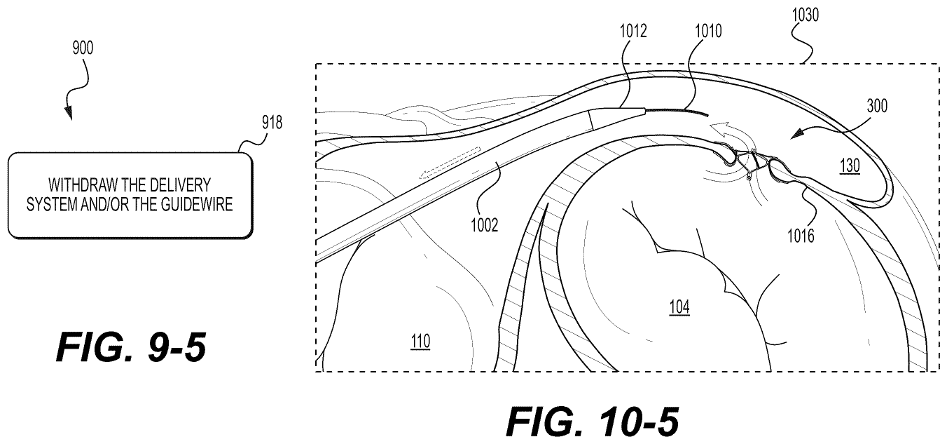

FIG. 3C illustrates a top view of the shunt of FIG. 3A according to one or more examples.

FIG. 4A illustrates a perspective view of a shunt in a configuration with an adjustable frame positioned within a portion of an anchoring frame according to one or more examples.

FIG. 4B illustrates a side view of the shunt of FIG. 4A according to one or more examples.

FIG. 4C illustrates a top view of the shunt of FIG. 4A according to one or more examples.

FIG. 5A illustrates a perspective view of an anchoring frame according to one or more examples.

FIG. 5B illustrates a side view of the anchoring frame of FIG. 5A according to one or more examples.

FIG. 5C illustrates a top view of the anchoring frame of FIG. 5A according to one or more examples.

FIG. 5D illustrates a flattened, rolled-out view of the anchoring frame of FIG. 5A as if the anchoring frame had been severed down the midline of an anchoring arm according to one or more examples.

FIG. 6A illustrates a perspective view of an adjustable frame according to one or more examples.

FIG. 6B illustrates a side view of the adjustable frame of FIG. 6A according to one or more examples.

FIG. 6C illustrates a top view of the adjustable frame of FIG. 6A according to one or more examples.

FIG. 6D illustrates a flattened, rolled-out view of the adjustable frame of FIG. 6A as if the adjustable frame had been severed down the middle line of a strut pattern according to one or more examples.

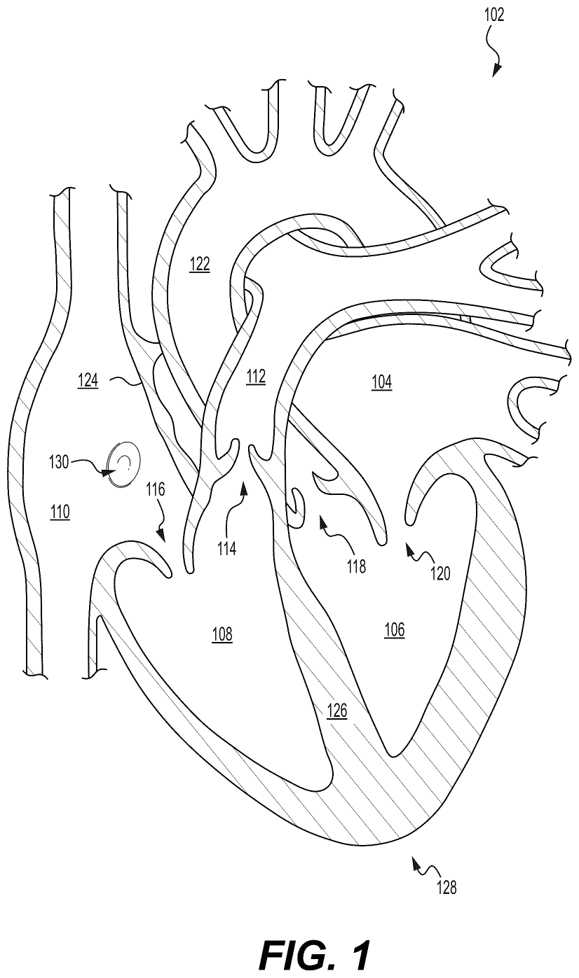

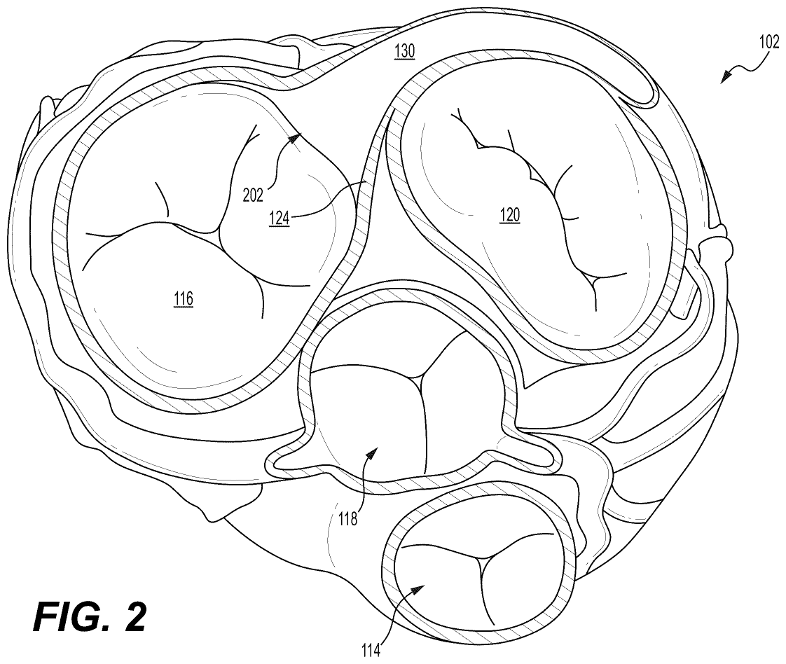

FIG. 7-1 illustrates a dual frame shunt including a covering disposed on an internal portion/surface of an adjustable frame according to one or more examples.

FIG. 7-2 illustrates a dual frame shunt including a covering disposed on an external portion/surface of an anchoring frame according to one or more examples.

FIG. 7-3 illustrates a dual frame shunt including a covering disposed on an external portion/surface of an anchoring frame and a covering disposed on an internal portion/surface of an adjustable frame according to one or more examples.

FIG. 8 illustrates various connection/attachment features that can be implemented to couple/attach an anchoring frame to an adjustable frame according to one or more examples.

FIGS. 9-1 through 9-5 illustrate a flow diagram illustrating a process for implanting a shunt in accordance with one or more examples.

FIGS. 10-1 through 10-5 illustrate images of cardiac anatomy and certain devices/systems corresponding to operations of the process of FIGS. 9-1 through 9-5 in accordance with one or more examples.

### DETAILED DESCRIPTION

The headings provided herein are for convenience only and do not necessarily affect the scope or meaning of the subject matter.

Although certain examples are disclosed below, subject matter extends beyond the specifically disclosed examples to other alternative examples and/or uses and to modifications and equivalents thereof. Thus, the scope of the claims that may arise here from is not limited by any of the particular examples described herein. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Certain standard terms of location are used herein to refer to certain device components/features and to the anatomy of animals, and namely humans, with respect to some examples. Although certain spatially relative terms, such as “outer,” “inner,” “upper,” “lower,” “below,” “above,” “vertical,” “horizontal,” “top,” “bottom,” “under,” “over,” “topside,” “underside,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

The term “associated with” is used herein according to its broad and ordinary meaning. For example, where a first feature, element, component, device, or member is described as being “associated with” a second feature, element, component, device, or member, such description can generally be understood as indicating that the first feature, element, component, device, or member is physically coupled, attached, or connected to, integrated with, embedded at least partially within, or otherwise physically related to the second feature, element, component, device, or member, whether directly or indirectly.

Certain examples of shunt implant devices are disclosed herein in the context of cardiac implant devices and cardiac physiology, which is discussed below in detail to provide context to aid in discussion of aspects of the devices disclosed herein. However, although certain principles disclosed herein are particularly applicable to the anatomy of the heart, shunt implant devices in accordance with the present disclosure may be implanted in, or configured for implantation in, any suitable or desirable anatomy.

### Cardiac Physiology

The anatomy of the heart is described below to assist in the understanding of certain features disclosed herein. In humans and other vertebrate animals, the heart generally comprises a muscular organ having four pumping chambers, wherein the flow between chambers and vessels associated therewith is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

FIGS. 1 and 2 illustrate vertical/frontal and horizontal/superior cross-sectional views, respectively, of an example heart 102 having various features/anatomy relevant to certain aspects of the present disclosure. The heart 102 includes four chambers, namely the left atrium 104, the left ventricle 106, the right ventricle 108, and the right atrium 110. In terms of blood flow, blood generally flows from the right ventricle 108 into the pulmonary artery 112 via the pulmonary valve 114, which separates the right ventricle 108 from the pulmonary artery 112 and is configured to open during systole so that blood may be pumped toward the lungs and close during diastole to prevent blood from leaking back into the heart from the pulmonary artery 112. The pulmonary artery 112 carries deoxygenated blood from the right side of the heart to the lungs.

In addition to the pulmonary valve 114, the heart 102 includes three additional valves for aiding the circulation of blood therein, including the tricuspid valve 116, the aortic valve 118, and the mitral valve 120. The tricuspid valve 116 separates the right atrium 110 from the right ventricle 108. The tricuspid valve 116 generally has three cusps or leaflets and may generally close during ventricular contraction (i.e., systole) and open during ventricular expansion (i.e., diastole). The mitral valve 120 generally has two cusps/leaflets and separates the left atrium 104 from the left ventricle 106. The mitral valve 120 is configured to open during diastole so that blood in the left atrium 104 can flow into the left ventricle 106, and, when functioning properly, closes during systole to prevent blood from leaking back into the left atrium 104. The aortic valve 118 separates the left ventricle 106 from the aorta 122. The aortic valve 118 is configured to open during systole to allow blood leaving the left ventricle 106 to enter the aorta 122, and close during diastole to prevent blood from leaking back into the left ventricle 106.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The atrioventricular (i.e., mitral and tricuspid) heart valves may further comprise a collection of chordae tendineae and papillary muscles (not shown) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae.

A wall of muscle, referred to as the septum, separates the left-side chambers from the right-side chambers. In particular, an atrial septum wall portion 124 (referred to herein as the “atrial septum,” “atrial septum,” or “septum”) separates the left atrium 104 from the right atrium 110, whereas a ventricular septum wall portion 126 (referred to herein as the “ventricular septum,” “interventricular septum,” or “septum”) separates the left ventricle 106 from the right ventricle 108. The inferior tip 128 of the heart 102 is referred to as the apex and is generally located on or near the midclavicular line, in the fifth intercostal space.

The coronary sinus 130 comprises a collection of veins joined together to form a relatively large vessel that collects blood from the heart muscle (myocardium). The ostium 202 (see FIG. 2) of the coronary sinus 130, which can be guarded at least in part by a Thebesian valve in some patients, is open to the right atrium 110, as shown. The coronary sinus 130 runs along a posterior aspect of the left atrium 104 and delivers less-oxygenated blood to the right atrium 110. The coronary sinus 130 generally runs transversely in the left atrioventricular groove on the posterior side of the heart.

As referenced above, certain physiological conditions or parameters associated with the cardiac anatomy can impact the health of a patient. For example, congestive heart failure is a condition associated with the relatively slow movement of blood through the heart and/or body, which causes the fluid pressure in one or more chambers of the heart to increase. As a result, the heart does not pump sufficient oxygen to meet the body's needs. The various chambers of the heart may respond to pressure increases by stretching to hold more blood to pump through the body or by becoming relatively stiff and/or thickened. The walls of the heart can eventually weaken and become unable to pump as efficiently. In some cases, the kidneys may respond to cardiac inefficiency by causing the body to retain fluid. Fluid build-up in arms, legs, ankles, feet, lungs, and/or other organs can cause the body to become congested, which is referred to as congestive heart failure. Acute decompensated congestive heart failure is a leading cause of morbidity and mortality, and therefore treatment and/or prevention of congestive heart failure is a significant concern in medical care.

The treatment and/or prevention of heart failure (e.g., congestive heart failure) can involve the shunting of some amount of fluid from a problematic high-pressure chamber/vessel to a lower-pressure chamber/vessel. For example, to address elevated left atrial pressure, a shunt can be implanted/formed in the interatrial septal wall or in the wall separating the left atrium from the coronary sinus to relieve pressure into the relatively lower pressure right atrium or the coronary sinus. Shunting can be achieved using an implanted shunt structure (e.g., stent or the like) or simply through formation of an opening/aperture in the septal wall. However, many shunt solutions are configured to permit a fixed amount of blood flow, which may result in too little or too much blood flowing between the chambers/vessels. Further, since the amount of shunting may not be precisely known before a procedure, the shunt may need to be replaced or another solution found if the shunt does not address the heart issue,

### Dual Frame Shunts

The present disclosure relates to systems, devices, and methods for shunting blood from a chamber/vessel of, for example, the heart to a relatively lower-pressure chamber/vessel using a dual frame shunt. The dual frame shunt can include two frame structures with different expansion/elasticity/deformation characteristics to facilitate adjustment of the amount of fluid that flows from one anatomical chamber/feature to another. The shunt can include an anchoring frame/structure that includes self-expanding characteristics and is configured to anchor the shunt within an opening between two anatomical chambers/features. The shunt can also include an adjustable frame/structure disposed outside or inside of at least a portion of the anchoring frame and configured to expand/adjust to various sizes. In some instances, the shunt can be delivered to a target site using a delivery catheter or another device/system for a minimally invasive procedure. The shunt can be compressed and coupled to the delivery catheter. The shunt can be released from the delivery catheter at a target site, wherein the anchoring frame can at least partially self-expand to secure the shunt to an opening between two anatomical chambers/features. The adjustable frame can be coupled to/contact the anchoring frame and configured to control an amount of blood that flows between the anatomical chambers/features. For instance, a dilation device can be used to set/adjust a size of a blood flow passage through the shunt. This can satisfy different blood flow needs to address heart or other issues more effectively (e.g., allow different amounts of pressure to be released from one chamber to another).

In many examples, the systems, devices, and methods are discussed in the context of shunting blood from the left side of the heart (e.g., the left atrium) to a relatively lower-pressure right chamber (e.g., right atrium, coronary sinus). Such shunting may be considered left-to-right shunting in that it involves the shunting of blood from a left-side chamber/vessel to a right-side chamber/vessel, which can be advantageous due to the higher fluid pressures typically experienced on the left (e.g., oxygenated) side of the blood circulation during at least portion(s) of the cardiac cycle. However, the systems, devices, and methods can be implemented in other contexts, such as for right-to-left shunting, other heart features, other types of fluids/gases, and/or other anatomical features.

FIGS. 3A-3C and 4A-4C illustrate views of an example dual frame shunt device/system 300 (also referred to as “the shunt 300”) that includes an anchoring frame/structure/component 310 (also referred to as “the self-expanding frame 310”) configured to anchor the shunt 300 to the desired anatomy and an adjustable frame/structure/component 320 configured to set/adjust an amount of shunting. FIGS. 3A-3C illustrate the adjustable frame 320 positioned/disposed outside of the anchoring frame 310 (sometimes referred to as “the external configuration”), while FIGS. 4A-4C illustrate the adjustable frame 320 positioned/disposed inside the anchoring frame 310 (sometimes referred to as “the internal configuration”). FIGS. 3A and 4A show perspective views of the shunt 300, FIGS. 3B and 4B show sides views of the shunt 300, and FIGS. 3C and 4C show top-down axial views looking through a central flow tube/lumen 302 formed by a barrel 304 of the shunt 300 (sometimes referred to as “the barrel structure 30 #) along an axis A₁ of the barrel 304. For ease of illustration in distinguishing the anchoring frame 310 from the adjustable frame 320, the adjustable frame 320 is sometimes shown with stippling in the figures.

FIGS. 3A-3C and 4A-4C illustrate the shunt 300 in an at least partially expanded form/configuration, such as a form when disposed/deployed within an opening between two anatomical features (e.g., chambers, vessels, etc.). When expanded, the barrel 304 and central flow lumen 302 of the shunt 300 can define a generally circular or oval opening for fluid passageway, as seen from above in FIGS. 3C and 4C, wherein the barrel 304 is configured to hold the sides of a puncture/hole in a tissue wall open and/or form a blood flow path between chambers/vessels on opposite sides of the tissue walls. The barrel 304 of the shunt 300 can refer to structure (e.g., walls, struts, wire, etc.) of the anchoring frame 310 and/or adjustable frame 320. In the external configuration (FIGS. 3A-3C), the barrel 304(A) of the anchoring frame 310 generally forms the blood flow path (e.g., is closest to the blood flow), while the barrel 304(B) of the adjustable frame 320 contacts the tissue within the opening. While in the internal configuration (FIGS. 4A-4C), the barrel 304(B) of the adjustable frame 320 generally forms the blood flow path and the barrel 304(A) of the anchoring frame 310 contacts the tissue within the opening. However, portions of the anchoring frame 310 and/or the adjustable frame 320 in either configuration can at least partially contact the tissue and/or form the blood flow path. In either configuration, the adjustable frame 320 and the anchoring frame 310 can be coupled together or contact each other such that the barrel 304(A) of the anchoring frame 310 is generally constrained/restricted by the adjustable frame 320, causing the amount of blood flow to be controlled/adjusted by the size of the adjustable frame 320, as discussed in further detail below.

The anchoring frame 310 can include a plurality of anchor arms 312(A)-312(D) (also referred to as “anchoring arms 312”) emanating from ends of the barrel 304. The anchor arms 312 can be any length and have tissue contact pads/feet 314(A)-314(D) associated with distal ends thereof, wherein such pads/feet 314 are configured to contact a surface of a tissue wall in which the shunt 300 is implanted. The illustrated example includes tissue circular/eyelet-type contact pads/feet 314, although other-shaped tissue-contact pads/feet may be implemented in connection with examples of the present disclosure. The pads/feet 314 can provide a relatively wide/spread-out area relative to the relatively narrow elongated strut/arm portions 312, which may distribute contact force/pressure exerted by the shunt 300 on the biological tissue wall/surface over a relatively wider area.

In the examples illustrated, the lower anchoring arms 312(C) and 312(D) each include an added eyelet 316, which can be attached to a suture/elongate member to assist in controlling the anchoring arms 312(C), 312(D) during deployment. For example, the sutures can extend out a proximal end of a delivery system and be pulled/pushed/controlled by a physician/user to collapse the anchoring arms 312(C), 312(D) and/or slowly release the anchoring arms 312(C), 312(D) (which can be deployed first at a target site, in some instances).

The sets of anchoring arms 312 can have shape memory configured to curl outward from the barrel 304 and/or axis A₁ (e.g., longitudinal axis) when deployed and/or expanded. Portions of such arms/anchor means, in the expanded configuration shown, can project approximately radially away from the imaginary reference axis A₁. Although four anchoring arms are illustrated, any number of arms can be implemented. In the example, the upper arm 312(A) is vertically offset from the anchor arm 312(B) (e.g., with respect to the vertical axis A₁), as seen in FIGS. 3A/4A and 4B/4B. However, the upper arm 312(A) may not be offset and/or any of the other arms may be offset vertically.

As noted above, the adjustable frame 320 can be configured/designed to be disposed around or inside the barrel 304(A) of the anchoring frame 310 and configured to control a size/diameter of a blood flow path through the shunt 300. For example, the adjustable frame 320 can be coupled to, contact/press against, or integral with the anchoring frame 310 to constrain/restrict the anchoring frame 310 from radial expansion. The adjustable frame 320 can generally be configured to adjust in size/diameter when more than a threshold external force is applied to the adjustable frame 320, such as by a dilation device. To constrain/restrict the anchoring frame 310, the adjustable frame 320 and the anchoring frame 310 can generally be designed such that a radial/expansion force exerted by the anchoring frame 310 (that is exerted to self-expand to a fully expanded form) is less than the threshold force needed to adjust the size/diameter of the adjustable frame 320.

To illustrate, in the external configuration (as illustrated in FIGS. 3A-3C), the adjustable frame 320 can prevent the barrel 304(A) of the anchoring frame 310 from expanding beyond an inner diameter D₁ of the adjustable frame 320 (as shown best in FIG. 3C), since the adjustable frame 320 surrounds the barrel 304(A) of the anchoring frame 310. Further, in the internal configuration (as illustrated in FIGS. 4A-4C), the adjustable frame 320 can be coupled to the barrel 304(A) of the anchoring frame 310 to prevent the anchoring frame 310 from expanding beyond the constraints imposed by being attached/coupled to the anchoring frame 310. In either configuration, the size/diameter of the adjustable frame 320 (e.g., D₁) is generally adjusted by a dilation device that is configured to be inserted into a lumen in the barrel 304 and exert a radially outward force.

The anchoring frame 310 and the adjustable frame 320 can include different characteristics to provide the various functionality for the shunt 300. For example, the anchoring frame 310 can be more elastic (less deformable) than the adjustable frame 320, thereby allowing the anchoring frame 310 to be delivered to a target site in a compressed/collapsed state and released to a predefined shape, while allowing the adjustable frame 320 to deform more easily and maintain a relatively more permanent form. In some instances, the anchoring frame 310 is formed of nitinol or another shape-memory/super elastic metal that can be compressed/deformed when an external force is applied and return to the pre-compressed/pre-deformed state when the external for is removed. Further, the adjustable frame 320 can be formed of stainless steel, cobalt chromium, aluminum, or another substance that can compress/deform when an external force is applied and maintain the compressed/deformed form when the external force is removed. In examples, the anchoring frame 310 has more elasticity than the adjustable frame 320 (e.g., the anchoring frame 310 includes a super elastic characteristic).

In examples, such as that shown in FIG. 3A, an opening/spacing between strut patterns of the adjustable frame 320 can provide a location for an anchoring arm 312 to extend. For instances, the anchoring arm 312(A) can extend between adjacent strut components (e.g., strut patterns/structures) of the adjustable frame 320, which can provide a more closely fit for the frames 310 and 320. In some instances (not shown), an anchoring arm 312 can extend through a central opening a strut pattern/structure.

Although various examples illustrate the adjustable frame 320 as a separate component/element from the anchoring frame 310, the adjustable frame 320 can be integral with the anchoring frame 310. For example, the anchoring frame 310 and the adjustable frame 320 can be implemented as a continuous/connected frame structure with different characteristics to implement the described functionality of the respective frames.

FIG. 5A-5D show various views of the anchoring frame 310 to illustrate examples details of the anchoring frame 310. FIG. 5A shows a perspective view of the anchoring frame 310, FIG. 5B shows a side view of the anchoring frame 310, FIG. 5C shows top-down axial view looking through a central flow tube/lumen of the anchoring frame 310, and FIG. 5D shows a flattened, rolled-out view of the anchoring frame 310 as if the anchoring frame 310 had been severed down the midline of the anchoring arm 312(A).

The anchoring frame 310 and/or barrel portion/structure 304(A) thereof can be defined at least in part by an arrangement of relatively thin struts/elements that form an array of cells/openings. For example, some or all of the anchoring frame 310 can be formed by super-elastic struts that are capable of compression into a delivery catheter and subsequent expansion back to the relaxed shape. Formation of the anchoring frame 310 using a plurality of interconnected struts forming cells therebetween can provide desirable flexibility of the anchoring frame 310, which enables compression and subsequent expansion at the implant site. The struts can be arranged in an interconnected pattern that omits any sharp corners or points, which might snag tissue when the shunt 300 is being manipulated/advanced through/within the puncture. The side walls of the barrel 304(A) together can define a tubular lattice that forms a channel for blood flow to pass through the shunt 300 when implanted. In examples, the anchoring frame 310 can be formed of a stent/stent-like wire frame structure. In the examples shown in FIGS. 5A-5D, the barrel 304(A) includes connection points/eyelets 502, which can be used to couple the anchoring frame 310 to the adjustable frame 320 and/or for visual marker features, as discussed herein.

The barrel 304(A) can be configured to be radially crimped to a collapsed or crimped state for introduction into the body on/in a delivery catheter and radially expandable to an expanded state for implanting the anchoring frame 310 at a desired location in the body (e.g., atrial septum, wall separating coronary sinus and left atrium, etc.). The anchoring frame 310 can be made of a plastically-expandable material that permits crimping of the struts thereof to a smaller profile for delivery and expansion, wherein such expansion may generally be achieved through self-expansion caused by shape memory/elasticity characteristics (although an expansion/dilation device can also be used, such as a balloon of a balloon catheter). For example, utilizing self-expanding struts/structure, the anchoring frame 310 can be crimped to a smaller profile and held in the crimped state with a restraining device, such as a sheath, covering the compressed anchoring frame 310. When the anchoring frame 310 is positioned at or near the target site, the restraining device can be removed to allow the anchoring frame 310 to be released and the anchoring frame 310 to self-expand to its expanded state. In an implanted state, the axis A₁ of the barrel 304(A) may be normal or askew/angled with respect to a line/plane that is normal to the tissue wall surface.

The anchoring arms 312 of the anchoring frame 310 can be angularly distributed about the axis A₁ of the shunt 300 and/or barrel 304 in any suitable or desirable manner. For example, as best illustrated in the axial view of FIG. 5C, the upper arms 312(A) and 312(B) can be angularly distributed in some manner about the perimeter/circumference of the barrel 304(A), wherein the upper arms 312(A) and 312(B), in the expanded configuration, project away from the barrel 304(A) and/or axis A₁ and are angularly offset by 180 degrees from each other, as indicated by the angle θ₁. Further, the lower arms 312(C) and 312(D) can be angularly offset from each other by a different amount, as indicated by the as indicated by the angle θ₂, and/or angularly offset from either of the upper arms 312(A) and 312(B), as indicated by the angle θ₃. In examples, such angular offset between the arms may serve to secure the anchoring frame 310 to the tissue wall in which is implanted in a manner as to resist post-implantation tilting, rotating, or other dislodging, migration, and/or undesirable movement of the shunt 100 from its desired implanted position and/or orientation. For example, the pressure contact points associated with the feet/pads 314 of the upper arms 312(A) and 312(B) on the distal side of the tissue wall in which the implant is disposed may be angularly offset from the tissue contact pressure points/areas associated with the feet/pads 314 of the lower arms 312(C) and 312(D), thereby distributing contact pressure over a wider area of the tissue wall in which the implant is disposed. In examples, the arms 312/feet/pads 314 can be covered in a sleeve/sock/covering (not illustrated). Although various angular offsets for the anchoring arms 312 are illustrated in the figures, the arms 30312 can be offset/positioned by other amounts.

In examples, one or more of the contact pads 314 and/or eyelets 502 can have associated therewith certain visual marker features configured to provide imaging visualization (e.g., increased visualization under imaging), such as ultrasound, x-ray, or other imaging modality. For example, the contact pads 314 and/or eyelets 502 (and/or other features of the anchoring frame 310 and/or adjustable frame 320) can include certain visual marker bands, studs, or the like comprising echogenic or other imaging-enhancement characteristics (e.g., radiopaque substances). In examples, a contact pad/eyelet can include an echogenic material, such as metal (e.g., tantalum) or other material configured to be relatively identifiable/visible under an imaging mechanism. The illustrated circular form of the various contact pads/eyelets associated with the respective anchor arms can facilitate a process for implementing the visual marker bands/studs therein, such as through a coining process or similar process. For example, a marker may be press-fit and/or melted/formed within the central opening/aperture of a contact pad/eyelet. Enhanced visualization features can aid in intraoperative placement of the shunt 300 in the target anatomy.

FIG. 6A-6D show various views of the adjustable frame 320 to illustrate examples details thereof. FIG. 6A shows a perspective view of the adjustable frame 320, FIG. 6B shows a side view of the adjustable frame 320, FIG. 6C shows top-down axial view looking through a central flow tube/lumen of the adjustable frame 320, and FIG. 6D shows a flattened, rolled-out view of the adjustable frame 320 as if the adjustable frame 320 had been severed down the midline of a strut pattern.

The adjustable frame 320 and/or barrel portion/structure 304(B) thereof can be defined at least in part by an arrangement of relatively thin struts/elements that form an array of cells/openings. Formation of the adjustable frame 320 using a plurality of interconnected struts forming cells therebetween can provide desirable expansion of the adjustable frame 320. The struts can be arranged in an interconnected pattern that omits any sharp corners or points, which might snag tissue when the shunt 300 is being manipulated/advanced through/within the puncture. In the examples shown in FIGS. 6A-6D, the struts form diamond shaped patterns; however, other shapes/patterns can be implemented, such as chevron patterns, wishbone patterns, crown-shaped patterns, etc. The side walls of the barrel 304(B) together can define a tubular lattice that forms a channel for blood flow to pass through the expandable frame 320 and/or to hold the tissue wall of within a puncture when implanted. For example, the interconnected struts around the barrel 304(B) can provide a cage-type structure that provides sufficient stability/integrity to hold the tissue at the puncture site open. In examples, the adjustable frame 320 can be formed of a stent/stent-like wire frame structure. In the examples shown in FIGS. 6A-6D, the barrel 304(B) includes connection points/eyelets 602 (e.g., at the top of the pages, relative to FIG. 6D, for example), which can be used to couple the adjustable frame 320 to the anchoring frame 310 and/or for visual marker features. The adjustable frame 320/barrel 304(B) can include a structure (e.g., wire frame) having a substantially cylindrical form. The length of the adjustable frame 320/barrel 304(B) L₁ (as seen in FIG. 6B) can be designed to fit/align with the anchoring frame 310.

The barrel 304(B) of the adjustable frame 320 can be configured to be radially expanded by an expansion/dilation device at an implant site (e.g., expand based on a radial force from a dilator). In examples, the barrel 304(B) is formed of a material that is configured to more permanently deform/expand, in comparison to the anchoring frame 310. For instances, the adjustable frame 320 can be formed of stainless steel, cobalt chromium, aluminum, or another material (e.g., deformable metal) that permits expansion of the adjustable frame 320 (when an external force is presented) and maintains the expanded/deformed shape when the external force is removed. In examples, the adjustable frame 320 is permanently deformable, such that the adjustable frame 320 is not configured to be compressed (at least without undergoing structural damage to the shape/form) after the adjustable frame 320 is expanded. To illustrate, the adjustable frame 320 can be configured to have an initial state in which the inner diameter D₁, as seen in FIG. 6C, has an initial value, wherein in the initial state the adjustable frame 320 is loaded onto a delivery device (e.g., coupled to a compressed anchoring frame 310). When released from the delivery device at the target site, the arms 312 of the anchoring frame 310 can self-expand. A dilation device can then be used to expand the adjustable frame 320 (e.g., increasing the diameter D₁), causing the barrel 304(B) of the anchoring frame 310 to expand as well. When the force from the dilation device is removed, the adjustable frame 320 can maintain the same increased diameter D₁. In some examples, the adjustable frame 320 is not configured to self-expand (e.g., is configured to expand only when an external force is applied).

In some examples, one or more components of the shunt 300 can be covered/attached to a covering/coating 702, as illustrated in FIGS. 7-1 through 7-3, which can be implemented to prevent corrosion and/or undesirable deformation due to frame components contacting each other directly and/or rubbing against each other. For instance, the anchoring frame 310 and the adjustable frame 320 may contact each other at one or more locations (which can cause the components to rub against each other when implanted within a patient). This may cause corrosion (e.g., galvanic corrosion) or other undesirable issues with the frame components. To prevent such issues, the covering 702 can be disposed between or attached to the anchoring frame 310 and/or the adjustable frame 320. The covering 702 can include a cloth (e.g., Polyethylene terephthalate (PET), woven textile, etc.), polymer/plastic (e.g., polyurethane), and/or any other material to provide a barrier/protection for the frame components. The covering 702 can be attached to one or more components of the shunt 300 in a variety of manners, such as through an adhesive, fastener, heat treatment (e.g., shrink-wrapping), etc.

In one example, as shown in FIG. 7-1, the covering 702 is disposed on an internal portion/surface of the adjustable frame 320, such as within the barrel 304(B) of the adjustable frame 320 to prevent the anchoring frame 310 from contacting the adjustable frame 320 directly. In another example, as shown in FIG. 7-2, the covering 702 is disposed on an external portion/surface of the anchoring frame 310. In a further example, as shown in FIG. 7-3, a first covering 702(A) is disposed on the anchoring frame 310 (e.g., external surface) and a second covering 702(13) is disposed on the adjustable frame 320 (e.g., internal surface). In yet other examples, the covering 702 can be implemented as a separate element that is positioned between the anchoring frame 310 and the adjustable frame 320 and held in place (without being attached to either frame structure) due to the frames pressing against each other.

Although FIGS. 7-1 through 7-3 illustrates the covering 702 positioned on the shunt 300 in the external configuration (where the adjustable frame 320 is positioned outside the anchoring frame 310), the covering 702 can similarly be implemented for the internal configuration (wherein the adjustable frame 320 is positioned within the anchoring frame 310). In the internal configuration, the covering 702 can similarly be disposed/positioned to prevent the anchoring frame 310 from directly contacting the adjustable frame 320. Further, in some cases, the covering 702 is positioned just at locations where the anchoring frame 310 contacts the adjustable frame 320.

As noted above, in examples, the anchoring frame 310 and the adjustable frame 320 are separate elements that are coupled/attached together. FIG. 8 illustrates various connection/attachment features 802(1)-801(N) (where N represents an integer) that can be implemented at various connection/attachment point 804 to couple/attach the anchoring frame 310 to the adjustable frame 320. Each connection point 804 can represent a position/point where the frames 310 and 320 contact each other and are connected or where the frames 310 and 320 are otherwise connected, such as in cases where there is spacing between the frames 310 and 320. Connection point 804(A) is representative of any of the connection points 804. The connection/attachment features 802 can include a variety of features, such as sutures 802(1), detent and cavity/receiving member 802(2), pin and hole/cavity 802(3), fastener 802(4), adhesive, crimped member/joint, weld (e.g., welded member/joint), solder, and so on. For ease of illustration, a few connection points 804 are illustrated. That is, other connection points can additionally, or alternatively, be implemented at various locations on the structure of the anchoring frame 310 and/or adjustable frame 320.

In some examples, one or more of the eyelets 502 of the anchoring frame 310 and/or eyelets 602 of the adjustable frame 320 can be coupled together using any of the connection/attachment features 802, as illustrated in the callout at 806. Although the eyelets 502 and 602 are not aligned in the illustration of FIG. 8 (or elsewhere), the anchoring frame 310 and the adjustable frame 320 can be positioned and/or the eyelets 502 and 602 can be designed such that the eyelets 502 and 602 are aligned (e.g., concentric), which can facilitate attachment/connection. However, the anchoring frame 310 and the adjustable frame 320 need not be aligned to facilitate attachment through a connection/attachment feature 802. Further, although FIG. 8 illustrates the shunt 300 in the external configuration, similar features 802 can be implemented for the internal configuration.

FIGS. 9-1 through 9-5 provide a flow diagram illustrating a process 900 for implanting a shunt device in accordance with one or more examples. FIGS. 10-1 through 10-1 through 10-5 provide images of cardiac anatomy and certain devices/systems corresponding to operations of the process 900 of FIGS. 9-1 through 9-5 in accordance with one or more examples. One or more of the acts/operations of the process 900 can be performed during the same medical procedure, separate medical procedures, or at other times.

At block 902, the process 900 includes providing a delivery system/device 1002 with the shunt implant 300 disposed therein in a delivery configuration. Image 1004 of FIG. 10-1 shows a partial cross-sectional view of a delivery/deployment system 1002 for the shunt implant device 300 in accordance with one or more examples of the present disclosure. Although a particular example of a delivery system is shown in various figures, shunt implant devices in accordance with aspects of the present disclosure may be delivered and/or implanted using any suitable or desirable delivery system and/or delivery system components.

The illustrated delivery system 1002 includes an inner catheter/shaft/sheath 1006, which may be disposed at least partially within an outer catheter/shaft/sheath 1008. In some examples, the shunt implant device 300 may be disposed at least partially around the inner catheter 1006 and at least partially within the outer sheath 1008. For example, the inner catheter 1006 may be disposed within the barrel portion 304 of the shunt implant 300. In some examples, the shunt implant 300 can be disposed about a shunt-holder portion/component, which may be integrated with the inner catheter/shaft 1006 or may be a separate component of the system 1002 that is attached or otherwise coupled to the inner catheter 1006 in some manner. In some examples, the shunt holder includes one or more cut-outs, indentations, recesses, channels, gaps, openings, apertures, holes, slits, or other features configured to accommodate the presence of the shunt implant 300 and/or other feature(s) or aspect(s) of the shunt implant 300.

In the compressed delivery configuration shown in image 1004, the shunt implant 300 can have a generally tubular form. The anchoring frame 310 can generally be compressed to fit within the outer sheath 1008, while the adjustable frame 320 can slide onto the anchoring frame 310/inner shaft 1006 in an initial non-dilated form. In such a configuration, the anchor features/arms 312 can form extensions of the barrel walls in a tubular form. For example, the tubular shape can correspond to a further-crimped configuration of the shape that the shunt 300 (e.g., anchoring frame 310) can have immediately after being laser cut from a tubular workpiece. During manufacturing, the un-crimped tubular form of the anchoring frame 310 can be deformed using mandrels and the like to bend the anchor features/arms radially outward into the expanded configuration, for example. The anchoring frame 310 in its deformed shape can then be heat treated such that the memory metal (e.g., nitinol) material reaches a transition temperature and the expanded shape becomes the relaxed/programmed shape of the anchoring frame 310. The memory metal shunt can then be bent into a tubular shape and crimped to decrease the diameter of the tube for loading within the delivery catheter 1002.

In some examples, the delivery system 1002 can be configured such that a guidewire 1010 can be disposed at least partially therein. For example, the guidewire 1010 can run in the area of an axis of the outer sheath 1008 and/or inner catheter 1006, as shown. The delivery system 1002 can be configured to be advanced over the guidewire 1010 to guide the delivery system 1002 to a target implantation site.

In some examples, the delivery system 1002 includes a tapered nosecone feature 1012, as shown, which may be associated with a distal end of the delivery system 1002. In some implementations, the nosecone feature 1012 can be utilized to dilate the opening in a tissue wall into which the shunt implant 300 is to be implanted, or through which the delivery system 1002 is to be advanced. The nosecone feature 1012 can facilitate advancement of the distal end of the delivery system 1002 through the tortuous anatomy of the patient and/or with an outer delivery sheath or other conduit/path. The nosecone 1012 can be a separate component from the catheter/inner shaft 1006 or may be integrated with the catheter 1006. In some examples, the nosecone 1012 is adjacent to and/or integrated with a distal end of the outer sheath 1008. In some examples, the nosecone 1012 may comprise and/or be formed of multiple flap-type forms that can be urged/spread apart when the shunt implant 300 and/or any portions thereof, the interior catheter 1006, or other device(s) are advanced therethrough.

The outer sheath 1008 can be used to transport the shunt implant 300 to the target implantation site. That is, the shunt implant 300 can be advanced to the target implantation site at least partially within a lumen of the outer sheath 1008, such that the shunt implant 300 is held and/or secured at least partially within a distal portion of the outer sheath 1008.

At block 904, the process 900 includes advancing the delivery system 1002 to a target implantation site. In one example, illustrated in image 1014 of FIG. 10-1, this includes advancing the delivery system 1002 into the coronary sinus 130 to a target implantation site adjacent a wall 1016 separating the coronary sinus 130 from the left atrium 104. Access to the target wall 1016 and left atrium 104 via the coronary sinus 130 may be achieved using any suitable or desirable procedure. For example, various access pathways can be utilized in maneuvering guidewires and catheters in and around the heart to deploy a shunt. In some examples, access can be achieved through the subclavian or jugular veins into the superior vena cava (not shown), right atrium 110, and from there into the coronary sinus 130. Alternatively, the access path can start in the femoral vein and through the inferior vena cava (not shown) into the heart. Other access routes can also be used, each of which can typically utilize a percutaneous incision through which the guidewire and catheter are inserted into the vasculature, normally through a sealed introducer, and from there the system may be designed or configured to allow the physician to control the distal ends of the devices from outside the body.

In some implementations, the guidewire 1010 is introduced through the subclavian or jugular vein, through the superior vena cava, and into the coronary sinus 130 via the right atrium 110. The guidewire 1010 can be disposed in a spiral configuration within the left atrium 104, which may help to secure the guidewire in place. Once the guidewire 1010 provides a path, an introducer sheath can be routed along the guidewire 1010 and into the patient's vasculature, such as with the use of a dilator. A deployment delivery catheter/system can be advanced through the superior vena cava to the coronary sinus 130 of the heart, wherein the introducer sheath can provide a hemostatic valve to prevent blood loss. In some examples, the deployment catheter can function to form and prepare an opening in the wall 1016 of the coronary sinus 130/left atrium 104 (e.g., create a puncture), and a separate placement delivery system, is used for delivery of the shunt implant 300. In other examples, the same system/catheter (e.g., the delivery system 1002) can be used as the both the puncture preparation and implant delivery with full functionality. The term “delivery system” can be used to represent a catheter or introducer with one or both of these functions.

In FIG. 9-2, at block 906, the process 900 includes advancing the delivery system 1002 through an opening 1018 formed in the wall 1016, as shown in image 1020 of FIG. 10-2. For example, the guidewire 1010 can be disposed as running through the opening 1018 prior to penetration thereof by the nosecone 1012 of the delivery system 1002. The opening 1018 can originally be formed using a needle (not shown) associated with the delivery system 1002 or another delivery system implemented prior to block 906. In some implementations, the nosecone feature 1012 can be used to at least partially dilate the opening 1018, which may have been previously dilated using a balloon dilator or other instrument.

At block 908, the process 900 includes deploying the shunt implant 300 using the delivery system 1002. For example, the outer sheath 1008 can be translated proximally/withdrawn while the inner sheath 1006 remains relatively fixed (e.g., removing the outer sheath 1008 to reveal the inner sheath 1006), thereby releasing one or more anchor arms 312 of the anchoring frame 310 on the atrial side of the wall 1016, as shown in image 1022 of FIG. 10-2. The outer sheath 1008 can continue to be translated proximally to deploy/place the rest of the shunt implant 300 (e.g., the adjustable frame 320) within the opening 1018 and release/deploy one or more other anchoring arms 312 of the anchoring frame 310 on the wall 1016 in the coronary sinus 130. The anchoring arms 312 can be configured to pinch/press against the wall 1016 in an opposing manner to hold the shunt implant 300 in place within the opening 1018.

The shunt implant 300 can allow blood flow to be shunted through the implant device 300 from the left atrium 104 into the right side of the heart via the coronary sinus 130. In examples, when the shunt implant 300 is initially deployed within the opening 1018, the dimeter of the adjustable frame 320 can generally be the same as when the shunt implant 300 is loaded onto the delivery system 1002, even though the anchor arms 312 of the anchoring frame 310 can be expanded/released to hold the shunt implant 300 in place. Here, the radially outward/expansion force of the barrel 304(A) of the anchoring frame 310 (when released from the delivery system 1002) will generally not be sufficient to change a diameter/size of the barrel 304(B) of the adjustable frame 320. Although the shunt implant 300 is shown in the left atrium/coronary sinus wall, the shunt implant 300 can be positioned between other cardiac chambers/vessels, such as between the left and right atria and/or other anatomical features.

In FIG. 9-3, at block 910, the process 900 includes setting an initial size of the shunt implant 300. For example, as shown in image 1024, a dilation device/instrument 1026 can be advanced/inserted over the guidewire 1010 (and/or through the delivery system 1002) and used to radially expand/dilate a lumen through a central portion of the shunt implant 300. This can expand/dilate the diameter of the adjustable frame 320 to establish/define/control an amount of blood flow that is shunted between the left atrium 104 and the coronary sinus 130. In the illustrated example, the dilation device 1026 includes a balloon/balloon catheter that is configured to expand radially, such as when positioned within the shunt implant 300. Further, the dilation device 1026 is shown as being implemented with the delivery system 1002. However, the dilation device 1026 can include other types of dilation devices and/or be implemented as a stand-alone device or with other systems.

At block 912, the process 900 can include determining a physiological parameter(s) (e.g., value of the parameter(s)) associated with the anatomical feature. This can include determining an amount/volume/velocity of blood flow between the left atrium 104 and the coronary sinus 130, determining blood pressure in the left atrium 104/coronary sinus 130/right atrium 110/another anatomical chamber/feature, a ratio of blood flow (e.g., volume, velocity, etc.) or pressure in one anatomical chamber/vessel to blood flow or pressure in another anatomical chamber/vessel, etc. In some examples, a sensor within a chamber(s)/vessel(s) can provide data/reading indicating such parameter(s) or data used to determine such parameter(s), wherein such sensor can be positioned on a distal end of the delivery system 1002/another device, positioned on the shunt implant 300, implanted within the anatomical chamber/feature, etc. Further, in some examples, echocardiography or another imaging technology can be implemented to determine a parameter(s). In yet further examples, other techniques can be used to determine a parameter(s). The parameter(s) is generally determined while the dilation device 1026 and/or the delivery system 1002 is removed from the anatomical chamber/feature, such as by removing the dilation device 1026/delivery system 1002 from the heart or patient, so that a more normal reading can be obtained. In some instances, the parameter(s) can be determined while the medical procedure is taking place (e.g., in the same procedure for implanting the shunt implant 300).

At block 914, the process 900 can include determining whether or not the parameter(s) satisfies one or more criteria (e.g., desired/predetermined pressure/volume/velocity). For example, the parameter(s) can be compared to a threshold(s), input into a function, etc. If the parameter(s) does not satisfy the one or more criteria (e.g., is less/greater than a threshold, etc.), the process 900 can proceed to block 916 in FIG. 9-4 (i.e., the “NO” branch). On this path, the shunt implant 300 is adjusted further to establish the appropriate amount of shunting. In contrast, if the parameter(s) satisfies the one or more criteria, the process 900 can proceed to block 918 in FIG. 9-5 (i.e., the “YES” branch).

In FIG. 9-4, at block 916, the process 900 can include adjusting a size of the shunt implant 300. For example, as shown in image 1028 of FIG. 10-4, the dilation device 1026 or another dilation device (e.g., that includes a larger balloon/dilation portion) can be inserted into the shunt implant 300 to further enlarge/dilate the shunt implant 300 (e.g., the adjustable frame 320), thereby increasing the amount of shunting between the left atrium 104 and the coronary sinus 130. The process 900 can the return to block 912 in FIG. 9-3 to determine the parameter(s) again (e.g., recalculate the value for the parameter(s)) and proceed to block 914). The process 900 can include making any number of adjustments to the shunt implant 300 (e.g., loops through block 916) to establish the appropriate amount of shunting for the specific context.

In FIG. 9-5, at block 918, the process 900 can include withdrawing the delivery system 1002 and/or the guidewire 1010. For example, as shown in image 1030 of FIG. 10-5, the delivery system 1002 and the guidewire 1010 can be translated proximally and/or removed from the patient. The shunt implant 300 can remain within the anatomy to provide shunting between the left atrium 104 and the coronary sinus 130.

Although described in the context of implanting the shunt 300 in the wall between the coronary sinus 130 and left atrium 104, the process 900 can be implemented, at least in part, to implant the shunt 300 in other anatomy and/or tissue walls, such as the atrial septum, ventricular septum, etc. The shunt 300 can also be positioned between other cardiac chambers/vessels, such as between the pulmonary artery and right atrium.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### Additional Features and Examples

1. A shunt system comprising: an anchoring frame configured to be disposed within an opening between a first anatomical feature and a second anatomical feature and expand from a collapsed state to an expanded state, the anchoring frame including a self-expanding form; and an adjustable frame disposed around at least a portion of the anchoring frame and configured to adjust in size to set an amount of fluid that flows between the first and second anatomical features.

2. The shunt system of any example herein, in particular example 1, wherein the anchoring frame includes one or more arms configured to, in the expanded state, extend from a central portion of the anchoring frame to anchor the shunt system to a tissue wall.

3. The shunt system of any example herein, in particular examples 1 or 2, wherein the adjustable frame comprises a permanently deformable form.

4. The shunt system of any example herein, in particular examples 1-3, wherein the anchoring frame includes a shape-memory material.

5. The shunt system of any example herein, in particular examples 1-4, wherein the adjustable frame includes at least one of stainless steel, cobalt chromium, or aluminum.

6. The shunt system of any example herein, in particular examples 1-5, wherein at least one of the anchoring frame or the adjustable frame includes a covering disposed between the anchoring frame and the adjustable frame.

7. The shunt system of any example herein, in particular examples 1-6, wherein the anchoring frame and the adjustable frame are independent frame structures that are coupled together with one or more attachment features. [0121] 8. The shunt system of any example herein, in particular examples 1-7, wherein the adjustable frame includes a wire frame having a substantially cylindrical form.

9. The shunt system of any example herein, in particular examples 1-8, wherein the anchoring frame includes a first anchoring arm configured to contact a first tissue wall within the first anatomical feature and a second anchoring arm configured to contact a second tissue wall within the second anatomical feature, the first anchoring arm being angularly offset from the second anchoring arm with respect to a longitudinal axis of the adjustable frame.

10. The shunt system of any example herein, in particular example 9, wherein at least one of the first anchoring arm or the second anchoring arm includes a visual marker configured to provide imaging visualization.

11. A method of providing blood flow between a first anatomical feature and a second anatomical feature, the method comprising: advancing a delivery catheter into an opening in a tissue wall between the first anatomical feature and the second anatomical feature; deploying, using the delivery catheter, a shunt within the opening, the shunt including an anchoring frame configured to anchor the shunt to the tissue wall and an adjustable frame disposed around at least a portion of the anchoring frame, the anchoring frame being configured with at least one of shape memory or a super elasticity characteristic; and dilating the adjustable frame to configure an amount of the blood flow between the first anatomical feature and the second anatomical feature.

12. The method of any example herein, in particular example 11, further comprising: determining at least one of the amount of blood flow between the first and second features or pressure in the first anatomical feature or the second anatomical feature; and adjusting the amount of blood flow by further dilating the adjustable frame.

13. The method of any example herein, in particular example 12, wherein the deploying the shunt, the dilating the adjustable frame, the determining, and the adjusting the amount of blood flow occur during the same medical procedure.

14. The method of any example herein, in particular examples 11-13, wherein the first anatomical feature is a left atrium of a heart and the second anatomical feature is at least one of a coronary sinus or a right atrium.

15. The method of any example herein, in particular examples 11-14, wherein the adjustable frame comprises a permanently deformable form.

16. The method of any example herein, in particular examples 11-15, wherein the adjustable frame includes at least one of stainless steel, cobalt chromium, or aluminum.

17. The method of any example herein, in particular examples 11-16, wherein at least one of the anchoring frame or the adjustable frame includes a covering disposed between the anchoring frame and the adjustable frame. [0131] 18. The method of any example herein, in particular examples 11-17, wherein the anchoring frame and the adjustable frame are independent frame structures that are coupled together with one or more attachment features.

19. The method of any example herein, in particular examples 11-18, wherein the deploying the shunt includes releasing a first arm of the anchoring frame to contact a first tissue wall within the first anatomical feature and releasing a second arm of the anchoring frame to contact a second tissue wall within the second anatomical feature.

20. A shunt system comprising: a first frame structure configured to anchor the shunt system to an opening between a first anatomical feature and a second anatomical feature, the first frame structure being configured to implement a compressed form on a delivery system and to self-expand to an at least partially expanded form when released from the delivery system; and a second frame structure coupled to at least a portion of the first frame structure, the second frame structure being configured to radially expand, based at least in part on a radial force from a dilator, to adjust an amount of fluid flow between the first and second features.

21. The shunt system of any example herein, in particular example 20, wherein the first frame structure includes a first anchoring arm configured to contact a first tissue wall within the first anatomical feature and a second anchoring arm configured to contact a second tissue wall within the second anatomical feature, the first anchoring arm being angularly offset from the second anchoring arm with respect to a longitudinal axis of the second frame structure.

22. The shunt system of any example herein, in particular examples 20 or

21. wherein the second frame structure comprises a permanently deformable form.

23. The shunt system of any example herein, in particular examples 20-22, wherein the second frame structure includes at least one of stainless steel, cobalt chromium, or aluminum.

24. The shunt system of any example herein, in particular examples 20-23, wherein at least one of the first frame structure or the second frame structure includes a covering disposed between the first frame structure and the second frame structure.

25. The shunt system of any example herein, in particular examples 20-24, wherein the first frame structure and the second frame structure are independent frame structures that are coupled together with one or more attachment features.

26. The shunt/shunt system of any example herein, in particular examples 1-25, wherein the shunt system is sterilized.

### Additional Examples

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the subject matter herein disclosed and claimed below should not be limited by the particular examples described above, but should be determined by a fair reading of the claims that follow.

Certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Terms (including technical and scientific terms) used herein can have the same meaning as commonly understood by one of ordinary skill in the art to which example examples belong. It be further understood that terms, such as those defined in commonly used dictionaries, can be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. The spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are generally intended to encompass the concepts of equality. For example, “less” can mean not only “less” in the strictest mathematical sense, but also, “less than or equal to.”

What is claimed is:

1. A shunt system comprising:
an anchoring frame configured to be disposed within an opening between a first anatomical feature and a second anatomical feature and expand from a collapsed state to an expanded state, the anchoring frame including a self-expanding form; and
an adjustable frame disposed around at least a portion of the anchoring frame and configured to adjust in size to set an amount of fluid that flows between the first and second anatomical features.

2. The shunt system of claim 1, wherein the anchoring frame includes one or more arms configured to, in the expanded state, extend from a central portion of the anchoring frame to anchor the shunt system to a tissue wall.

3. The shunt system of claim 1, wherein the adjustable frame comprises a permanently deformable form.

4. The shunt system of claim 1, wherein the anchoring frame includes a shape-memory material.

5. The shunt system of claim 1, wherein the adjustable frame includes at least one of stainless steel, cobalt chromium, or aluminum.

6. The shunt system of claim 1, wherein at least one of the anchoring frame or the adjustable frame includes a covering disposed between the anchoring frame and the adjustable frame.

7. The shunt system of claim 1, wherein the anchoring frame and the adjustable frame are independent frame structures that are coupled together with one or more attachment features.

8. The shunt system of claim 1, wherein the adjustable frame includes a wire frame having a substantially cylindrical form.

9. The shunt system of claim 1, wherein the anchoring frame includes a first anchoring arm configured to contact a first tissue wall within the first anatomical feature and a second anchoring arm configured to contact a second tissue wall within the second anatomical feature, the first anchoring arm being angularly offset from the second anchoring arm with respect to a longitudinal axis of the adjustable frame.

10. The shunt system of claim 9, wherein at least one of the first anchoring arm or the second anchoring arm includes a visual marker configured to provide imaging visualization.

11. A method of providing blood flow between a first anatomical feature and a second anatomical feature, the method comprising:
advancing a delivery catheter into an opening in a tissue wall between the first anatomical feature and the second anatomical feature;
deploying, using the delivery catheter, a shunt within the opening, the shunt including an anchoring frame configured to anchor the shunt to the tissue wall and an adjustable frame disposed around at least a portion of the anchoring frame, the anchoring frame being configured with at least one of shape memory or a super elasticity characteristic; and
dilating the adjustable frame to configure an amount of the blood flow between the first anatomical feature and the second anatomical feature.

12. The method of claim 11, further comprising:
determining at least one of the amount of blood flow between the first and second features or pressure in the first anatomical feature or the second anatomical feature; and
adjusting the amount of blood flow by further dilating the adjustable frame.

13. The method of claim 12, wherein the deploying the shunt, the dilating the adjustable frame, the determining, and the adjusting the amount of blood flow occur during the same medical procedure.

14. The method of claim 11, wherein the deploying the shunt includes releasing a first arm of the anchoring frame to contact a first tissue wall within the first anatomical feature and releasing a second arm of the anchoring frame to contact a second tissue wall within the second anatomical feature.

15. A shunt system comprising:
a first frame structure configured to anchor the shunt system to an opening between a first anatomical feature and a second anatomical feature, the first frame structure being configured to implement a compressed form on a delivery system and to self-expand to an at least partially expanded form when released from the delivery system; and
a second frame structure coupled to at least a portion of the first frame structure, the second frame structure being configured to radially expand, based at least in part on a radial force from a dilator, to adjust an amount of fluid flow between the first and second features.

16. The shunt system of claim 15, wherein the first frame structure includes a first anchoring arm configured to contact a first tissue wall within the first anatomical feature and a second anchoring arm configured to contact a second tissue wall within the second anatomical feature, the first anchoring arm being angularly offset from the second anchoring arm with respect to a longitudinal axis of the second frame structure.

17. The shunt system of claim 15, wherein the second frame structure comprises a permanently deformable form.

18. The shunt system of claim 15, wherein at least one of the first frame structure or the second frame structure includes a covering disposed between the first frame structure and the second frame structure.

19. The shunt system of claim 15, wherein the first frame structure and the second frame structure are independent frame structures that are coupled together with one or more attachment features.

20. The shunt system of claim 15, wherein the shunt system is sterilized.
